(19) United States
(12) Patent Application Publication
(10) Pub. No.: US 2008/0275105 A1
(43) Pub. Date: Nov. 6, 2008

(54) Selective Muscle Relaxant and Pharmaceutical Compositions

(75) Inventors: Carlos Alberto Manssour Fraga, Rio de Janeiro (BR); Eliezer de Jesus de Lacerda Barreiro, Rio de Janeiro (BR); Arthur Eugen Kummerle, Rio de Janeiro (BR); Alexandre Godinho Silva, Rio de Janeiro (BR); Roberto Takashi Sudo, Rio de Janeiro (BR); Gisele Zapata-Sudo, Rio de Janeiro (BR)

Correspondence Address: SMITH, GAMBRELL & RUSSELL, SUITE 3100, PROMENADE II, 1230 PEACHTREE STREET, N.E., ATLANTA, GA, 30309-3592, US

(73) Assignee: UNIVERSIDADE FEDERAL DO RIO DE JANEIROUFRJ, Ilha do Governador (BR)

(21) Appl. No.: 11/573,914

(22) PCT Filed: Aug. 19, 2005
(86) PCT No.: PCT/BR05/00168

§ 371 Date: Feb. 19, 2007

(30) Foreign Application Priority Data
Aug. 20, 2004 (BR) P1040.3363-9

(51) Int. Cl. A61K 31/381; C07D 409/12; A61P 21/02

(52) U.S. Cl. 514/444; 549/60

(57) ABSTRACT

This invention refers to substances able to cause selective muscle relaxation, pharmaceutical compositions containing such compounds and their use in the treatment of muscle tissue diseases, with such compounds complying with the general formula (I).

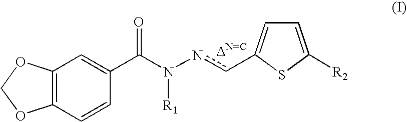

### FIELD OF THE INVENTION

This invention encompasses compounds that can act as selective muscle relaxants, with these compounds belonging to the thienyl-acylhydrazone family. This invention also covers pharmaceutical compositions containing such compounds and their use in the treatment of muscle tissue diseases.

### BACKGROUND OF THE INVENTION

New bioactive substances belonging to the N-acylhydrazone class were synthesized using material taken from safrole, a natural Brazilian product obtained from oil of sassafras (Ocotea pretiosa) (Figueiredo & cols., 2000). A probable pharmacological property of this class would be an analgesic effect, as the hydrazone link in the molecules plays an important role in cyclo-oxygenase inhibition (Todeschini & cols., 1998). Replacing the furoyl ring by the N-heteroaryl-hydrazone molecule thiophene gave rise to 3,4-methylenedioxybenzyl-2-thienylhydrazone, shown in the structure below, called LASSBio 294 (L294) and identified as a type 4 phosphodiesterase (PDE) inhibitor bioisoster (Piaz & cols., 1997), an enzyme isoform responsible for cyclic nucleotide degradation.

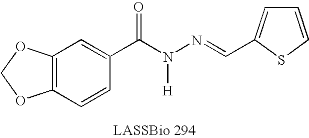

The L294 was described as a positive inotropic cardiac agent (Sudo & cols., 2001) with vasodilatory property (Silva & cols., 2002), and also able to increase skeletal musculature contractility in frogs (Gonzalez-Serratos & cols., 2001) and rats (Zapata-Sudo & cols., 2003). The increased cardiac contractility caused by L294 was not related to β-adrenergic receptor activation, as the increase in the range of the muscle twitch was not modified through prior treatment with propranolol, a non-selective blocker for these receptors. The possibility that L294 may be increasing the sensitivity of the contractile proteins to Ca²⁺ in the cardiac muscle was discarded, due to the lack of alterations to the [Ca²⁺] curve versus tension in denuded cardiac fibers (Sudo & cols., 2001). The probable action mechanism of L294 was assigned to the greater build-up of Ca²⁺ in the sarcoplasmic reticulum (SR) due to the greater uptake of Ca²⁺ by this organelle. We cannot eliminate the possibility of PDE inhibition increasing the adenosine cyclic monophosphate (cAMP), which would result in a higher uptake of Ca²⁺ by the SR through the phospholamban phosphorylation found in the Ca²⁺-ATPase pump (SERCA, “sarcoplasmic reticulum Ca-ATPase”).

The L294 also reverted to a contracture of the aorta rings induced as much by noradrenaline as by KCl (Silva & cols., 2002), which effect is observed in whole endothelium preparations. The involvement of the release of prostanoids by the L294 to explain the vascular relaxation effect was discarded as the relaxation remained unaltered, even in the presence of a cyclo-oxygenase inhibitor. The reversal of the relaxation by the guanylate cyclase inhibition suggested the involvement of cGMP in the L294 mechanism for promoting vasodilation.

As the physiological function of the PDE is related to both the increased contractility of the cardiac muscle as well as the relaxation of the vascular smooth muscle, this important pharmacological target is of much interest in the quest for new substances that act through interfering with the PDE function in different muscle tissues.

Cardiac Muscle

The muscle excitation/contraction coupling is a process that begins with the generation of a potential action (PA) that spreads along the sarcolemna and culminates with the increase in the Ca²⁺ intracellular concentration. Cardiac muscle excitation involves alterations in the ion permeability of the cell membrane, which initially allows the influx of Na⁺ followed by the activation of the Ca²⁺ channels, type L or sensitive to dihydropyridines (DHPR). The influx of Ca²⁺ through the DHPR, together with the release of the Ca²⁺ intracellular stores are responsible for the increased Ca²⁺ concentration in the cytosol, which causes muscle contraction through linking up with the contractile proteins. Consequently, the myofilaments are activated by the Ca²⁺ deriving from the combination of the influx through the sarcolemna and the release by the SR through the ryanodine (RyR2) and/or the inositol 1,4,5-triphosphate (IP₃R) channels.

The SR is an organelle that stores Ca²⁺ through cytoplasmic Ca²⁺ uptake by an ATP-dependent carrier, Ca²⁺-ATPase, SERCA. The main SERCA2a isoform found in the cardiac cells that actively carry Ca²⁺ of the cytoplasm to the SR lumen, is a pump regulated by an intrinsic protein known as phospholamban. This protein reduces the affinity of the SERCA2a for the Ca²⁺, and, when phosphorylated, consequently switches off and ceases to inhibit the ion uptake. The high level of Ca²⁺ stored in the SR is the main factor behind the increase in the free concentration of the ion in the sarcoplasm. The cytoplasm release is handled mainly through the ryanodine channel, a tetrameric structure (Block & cols. 1988; Saito & cols., 1988) located at the junction between the SR and the T-tubule, where it is linked to the type L Ca²⁺ channels (Carl & cols., 1995; Sun & cols., 1995). The RyR in the SR of any muscle tissue is given this name due to its affinity for the alkaloid ryanodine (Stern & cols., 1992). This is closely related to other proteins such as phosphatases, calmodulin, calsequestrin and FKBP12 and can be activated by caffeine, Ca²⁺ and ATP and inhibited by Mg²⁺, procaine and red ruthenium. Another type of channel that activates Ca²⁺ release is the IP₃R, sensitive to the IP₃ second messenger produced by inositol phosphate degradation by the phospholipase C (PLC). When activated, the α₁-adrenergic receptors increase the inotropism, through the IP₃.

The SR may be divided into two parts: 1) The longitudinal tubules system running parallel to the myofilaments and ending in large chambers called junction or terminal cisterns. In this region, ryanodine channels predominate with a large quantity of calsequestrin; 2) the sarcotubular network, rich in Ca²⁺ pumps and located centrally, in closer contact with the contractile system.

The important events for reducing Ca²⁺ at the end of the muscle contraction are essentially pumping the Ca²⁺ back to the SR and extruding it into the extracellular medium. At the end of the contraction, the intracellular concentration of Na⁺ is increased, which favors the functioning of the Na⁺/K⁺-ATPase, that re-establishes the Na⁺ concentration gradient, expelling three Na⁺ ions against the entry of two K⁺ ions. The reduction in Na⁺ in the cardiac myocyte results in the activation of the Na⁺/Ca²⁺ exchanger in the plasma membrane, expelling the Ca²⁺ from the cytoplasm. Another way through which the Ca²⁺ concentration can return to the repose value is through the Ca²⁺ efflux performed by the PMCA type Ca²⁺-ATPase pump. This pump in the sarcolemna works through expending the energy derived from the ATP hydrolysis, having a high affinity with Ca²⁺ and low transportation speed.

Consequently, the Ca²⁺ plays a leading role in regulating the contraction and relaxation of the cardiac muscle, and may be found free in the cytosol or stored in intracellular structures such as the mitochondrias and the SR. Alterations to the Ca²⁺ concentration in any of these compartments may affect the contraction and/or relaxation processes of the cardiac musculature. Some substances such as the β-adrenergic receptor agonists may increase cardiac contractility by increasing the intracellular concentration of Ca²⁺. They increase the production of cAMP that in turn activates a cAMP dependent protein kinase that causes the phosphorylation of the type L Ca²⁺ channel and the FKBP12 in the ryanodine channel, which allows the influx of extracellular Ca²⁺ and the mobilization of the intracellular stores (Kuschel & cols., 2000). The description of the cAMP as a second messenger in response to epinephrine paved the way for understanding the physiological functions of the nucleotides and the phosphodiesterases (PDE) on adrenergic activation in different tissues. Since then, it has prompted interest through describing the intracellular paths of action able to explain the effects produced by hormones and other molecules that act through nucleotides (Beavo & Brunton, 2002). The PDE is a protein that is soluble in the cytoplasm with enzymatic activity that can separate the phosphodiester link of the nucleotides, making them inactive; additionally, it intervenes in the duration and intensity of the nucleotide effects. Nineteen genes have already been described, subgrouped into eleven distinct PDE families (Sordeling & Beavo, 2000), based on the primary sequence of aminoacids and catalytic and regulatory sites, with each family containing multiple genes that code its variations. Expressing this enzyme is closely linked to the specific characteristics of the tissue, making the synthesis of new selective PDE inhibitors of clinical interest for modulating certain intracellular events in different tissues. In addition to the catalytic sites, the PDE has 270 amino acids, with more than 50% homology among the isoforms, a moderate homology regulatory site able to link different modulators among the existing families.

In the ventricular myocyte, the most abundant isoforms are the PDE2 and the PDE4 (Verde & cols., 1999), with the PDE1 and the PDE3 also being found, and the PDE9 described more recently. Isoforms 1 and 2 have similar affinities for the cAMP and the cGMP, being activated by complex Ca⁺²-calmodulin and cGMP respectively. Isoforms 3 and 4 have greater affinity for the cGMP, with the PDE4 being activated by cAMP-dependent phosphorylation (Table 1). The inhibition of isoforms 2, 3 and 4 (Verde & cols., 1999) can increase the Ca²⁺ basal current, resulting in better cardiac contractility.

As described above, the cAMP plays an important role in cardiac contractility through cAMP-dependent protein kinase, increasing the probability of opening the type L Ca⁺² channel. Along the same lines, the nucleotide can foster ion removal through activating the Ca²⁺-ATPase pump.

Cardiac inotropism may be modulated through the use of drugs that act on these important targets listed above, which are responsible for increasing the intracellular Ca²⁺ and the sensitivity of the contractile proteins to this ion. The oldest drugs with this property are the cardiac glycosides, also known as digitalics as their active principle comes from plants belonging to the Digitalis genus (digoxin and digitoxin). These directly inhibit the Na⁺/K⁺-ATPase pump (Akera & Brody, 1997), increasing the intracellular concentration of Na⁺, with a consequent reduction in the Ca²⁺ efflux through the Na⁺/Ca²⁺ exchanger. The greater retention of Ca²⁺ results in an increase in the intracellular stores with a larger quantity of Ca²⁺ to be released. In this case, during the contraction event, there is a greater availability of Ca²⁺ to interact with the contractile proteins, which characterizes the positive inotropism of these drugs. The cardiotonic agents cause adverse effects related to excess intracellular Ca²⁺, such as cardiac arrhythmias and injuries that may result in cell death.

Another class of inotropic agents includes drugs that act on the contractile proteins, increasing the link between the Ca²⁺ to the troponin C (TnC) at the thin filament regulatory sites and/or directly in the cross-point cycle (Endoh, 1998; Lee & Allen, 1997). These drugs are known as Ca²⁺ sensitizers and interest in them is related to the fact that they do not present the same toxicity as the cardiotonics. The link between two Ca²⁺ ions and TnC weakens the link between the Tnl and actin; shifting the tropomyosin from its site allows the actin to interact with the myosin, shortening the muscle fibers and resulting in muscle contraction (Katz, 2061). The Ca²⁺ sensitizers may be divided into three classes: Class 1) they increase the length between the Ca²⁺ and the troponin C; Class 2) they facilitate actin regulation through the Ca²⁺ complex with the thin filaments; Class 3) they act directly on the actin-myosin interaction.

Levosimendan and pimobendan are examples of Ca²⁺ sensitizer agents that cause positive cardiac inotropism in vitro and in vivo for animal models (Endoh, 1995) through increase in the sensitivity of the contractile proteins to the Ca²⁺. In addition to reducing the Ca²⁺-dependent actin displacement threshold (Sata & cols., 1995), a Class 1 agent, pimobendan, also inhibits the PDE3 (Scholz & Meyer, 1986). A Class 2 agent, levosimendan acts mainly through stabilizing the conformation of the Ca⁺²-troponin C complex, in addition to PDE3 inhibition.

Vascular Smooth Muscle

Like other muscle cells, the contraction of the vascular smooth musculature depends on increasing the intracellular concentration of Ca²⁺, which takes place by an influx through opening the ion channels in the plasma membrane or through release from the intracellular stores. The electrochemical and pharmacological coupling (FIG. 3) are responsible for altering the Ca²⁺ concentration. For the former, the generation of a PA, where the membrane potential shifts from −60 mV to 10 mV opens up the voltage-dependent Ca²⁺ channels allowing the influx of Ca²⁺, which favors the electrochemical gradient. These ion channels are complex proteins with specialized regions sensitive to changes in the membrane potential and cause alterations to the channel conformation, which allow the influx of Ca²⁺. For the pharmacological coupling, the Ca²⁺ concentration may be altered without changing the membrane potential, meaning the Ca²⁺ influx (Nelson & cols., 1988) and its intracellular stores mobilization (Hashimoto, T., 1986) may occur through linking extracellular agents to the membrane receptors. Through its link to the membrane receptors coupled to the stimulatory protein G, noradrenaline activates the formation of second messages through the phospholipase C (PLC) that can control the muscle contraction process. One of these messengers is the IP3, whose intracellular molecule activates the release of Ca²⁺ from the SR. The influx of Ca²⁺ can mobilize its intracellular stores (Herrmann-Frank & cols., 1991; Nelson & cols., 1995) and increase its concentration in the cytoplasm. Through the action of the PLC, diacylglycerol (DAG) is also formed, whose role is to increase the sensitivity of the myofilaments to the Ca²⁺.

Invaginations in the muscle fibers called caveolae (Taggart, 2001) play a regulatory function in the Ca²⁺ homeostasis. When stimulated, the caveolin proteins forming the invaginations recruit enzymes from the outskirts of the cell involved in the inhibition of the myosin phosphatase, which is the enzyme responsible for the dephosphorylation of the light myosin chain, an important stage in the muscle relaxation process.

At the end of the contraction stimulation, the relaxation of the smooth musculature does not occur immediately, with a reduction in the Ca²⁺ concentration and myofilament dephosphorylation, being the means through which relaxation occurs. The same SR that releases Ca²⁺ in the contraction process is the main factor responsible for the ion reabsorption against the concentration gradient, thanks to the Ca²⁺-ATPase (SERCA pumps) found largely exposed to the cytoplasm. The relaxation of the smooth musculature may be induced pharmacologically through cyclic nucleotide generation or blocking the degradation of this second messenger. Another path to relaxation is related to activating the voltage-dependent potassium channel that, once open, causes cellular repolarization, which is an event triggering a reduction in the intracellular Ca²⁺.

The activation of the beta-adrenergic receptors in tracheal vascular smooth muscle cells induced by agonists such as salbutamol, albuterol and phenoterol, leads to cAMP formation, the second messenger that is responsible for carrying the Ca²⁺ back to the SR through protein kinase, causing relaxation (Hoiting & cols., 1996).

The vascular endothelial cells are responsible for the formation of molecules that can lead to the relaxation of the vascular smooth muscle. The increase in Ca²⁺ caused by the muscarinic agonists such as ipratropium, for example can activate nitric oxide (NO) synthesis through NO syntasis. The NO spreads through the vascular smooth muscle and promotes the formation of cGMP in the intracellular medium through guanylate cyclase (Ijioma & cols., 1995), which in turn acts on the smooth musculature relaxation, activating the myosin phosphatase, which is the enzyme responsible for the dephosphorylation of the light myosin chain. Through a cGMP-dependent kinase enzyme, the nucleotide reduces the cytoplasmic Ca²⁺ by activating the SERCA and PMCA pumps. The phospholamban phosphorylation in the Ca²⁺-ATPase of the SR activates this pump (Raeymaekers, 1988) just as the kinase enzyme activates the pump in the plasma membrane (Yoshida, 1999)

The contractile activity of the smooth musculature is also a PDE inhibitor target. Selective molecules for the vascular smooth muscle isoforms may be used to treat vascular and erectile dysfunctions.

In the vascular smooth muscle, from 1 to 5 isoforms were identified (Saeki & Saito, 1993). A selective PDE inhibitor for the type 3 isoform, the milrinone has a positive cardio-inotropic effect and peripheral vasodilator effect that result in some hemodynamic effects such as a reduction in the post-load with an increase in the cardiac debit and a reduction in the peripheral vascular resistance (Shipley & cols., 1996).

In the vascular smooth muscle of the corpus cavernosa, the inhibition of the principal isoform, PDE5, increases the supply of cGMP that, through cGMP-dependent protein kinase, reduces the probability of opening the Ca²⁺ membrane channels, resulting in the relaxation of the tissue responsible for the erection (Archer, 2002). This is how drugs such as sildenafil, vardenafil and tadalafil act.

Skeletal Muscle

In the skeletal muscle, the contraction process begins with the depolarization of the nerve endings of the neuromuscular junction with the release of acetylcholine and activation of the nicotine receptors in the muscle fiber. The neurotransmitter increases the permeability of the fiber to the Na⁺ ions in the chemo-excitable regions (motor plaque) generating the motor plaque potential (MPP). Depolarization focused on the motor plaque excites the adjacent region (electro-excitable membrane). In this region, the influx of Na⁺ occurs, giving rise to the all-or-nothing type PA that propagates along the T-tubule. In contrast to the cardiac muscle, the increase in the intracellular Ca²⁺ arising from the release of Ca²⁺ by the SR does not depend on the influx of this ion from the extracellular medium. The depolarization of the muscle fiber membrane (T-tubule) is perceived by the DHPR (McPherson & cols., 1993) and transmitted to the RyR1 channel that in turn causes the release of Ca²⁺ from the SR to the cytosol. Once activated, the RyR1 transmits information that releases the Ca²⁺ binding of the calsequestrin, a protein found in the SR lumen (Ikemoto & cols., 1991). The release of the Ca²⁺ by the skeletal muscle SR may be activated by pharmacological agents such as caffeine, halothane, ryanodine (nanomolar concentration), adenine nucleotides, 4-chloro-m-cresol and others. The ATP and the Ca²⁺ are the main endogenous activators. This may be blocked by Mg²⁺ ryanodine (micromolar concentration), red ruthenium, procaine, calmodulin, dantrolene and others.

Patent literature contains some quite relevant documents. We may mention WO 00/78754, which gave rise to the LASSBio 294 compound, describing compounds with inotropic capacity. However, in contrast with the purpose of this invention, these state-of-the-art compounds are considered as positive inotropics.

Compounds able to cause selective muscle relaxation are not unknown. We may mention document WO 94/28902, which describes compounds able to act selectively on the vascular smooth muscle. The WO 04/050084 document reveals new compounds with a capacity to inhibit ACE and be NO donors at the same time. The WO 04/047837 document discloses new multifunction β-adrenergic blockers with additional capacities, such as NO donors and anti-oxidizing action.

However, no compound described previously presents structural and pharmacodynamic similarities to the compounds of this invention.

### DESCRIPTION OF THE FIGURES

FIG. 1 shows the typical record of muscle twitches in rat papillary muscle, stimulated electrically at a frequency of 1 Hz. The lines were obtained for the control, 30 minutes after the balance of the preparation in the presence of 100 μM of the L789, L791 and L786 derivatives and 30 minutes after bathing the tissues with Tyrode's solution.

FIG. 2 shows the effects of the N-acylhydrazone derivatives in the range of the papillary muscle twitches in rats. The data represents the average ±SEM of six experiments for each derivative tested. (*p<0.05 when compared to the control).

FIG. 3 shows the effects of the N-acylhydrazone derivatives on the isometric tension in the left ventricle bundles of a rat heart. The data represent the average ±SEM of six experiments for each derivative tested. (*p<0.05 when compared to the control).

FIG. 4 shows the record representing the Protocol Experiment used to test the relaxing activity of the smooth musculature of the aorta, with the endothelium preserved by the N-acylhydrazone derivatives. In the example, the tested substance was L785, which caused full relaxation of the contracture induced by phenylephrine (PNP). The arrows indicate the moments when the indicated substances were added to the nutrient solution. In the records, note bathing the preparation in Tyrode's solution for thirty minutes.

FIG. 5 shows the inhibition of the contracture induced by the phenylephrine through the L294 analogs in rat aorta rings with the vascular endothelium preserved. The points represent the average of SEM of six experiments for each derivative. (*p<0.05 in relation to the control).

FIG. 6 shows the dose-response curve for the cumulative concentrations of the derivatives on the aorta ring contracture induced by KCl (40 mM). (*p<0.05 when compared to the control. n=6).

FIG. 7 shows the record representing the Protocol Experiment used to test the relaxing activity of the smooth musculature of the aorta, without the endothelium, by the N-acylhydrazone derivatives. In the example, the substance tested was L790 which did not cause a significant relaxing effect on the contracture induced by phenylephrine (PNP). The arrows indicate the moments at which the indicated substances were added to the nutrient solution. In the records, note bathing the preparation in Tyrode's solution for thirty minutes.

FIG. 8 shows the dose-contractile response curve for the aorta rings in the presence of N-acylhydrazone derivatives. The dots represent the average ±SEM of six experiments for each derivative. (* indicates p<0.05 when compared to the control).

FIG. 9 shows the inhibitory effect of the L294 analogs on the contracture of rat aorta rings with no endothelium, induced by KCl (40 mM). The dots represent the average ±SEM (n=6). (*p<0.05 in relation to the control).

FIG. 10 shows the effect of accumulated concentrations of the L786 derivative on the contracture in the corpus cavernosa of a male guinea pig induced by phenylephrine (30 μM).

FIG. 11 shows the effect of accumulated concentrations of the L294 derivative on phenylephrine-induced contracture in the corpus cavernosa of a male guinea pig. * indicates p<0.05 when compared to the control. The numbers in brackets correspond to the number of experiments carried out.

FIG. 12 shows the effect of accumulated concentrations of the L785 derivative on the contracture of the tracheal rings of a rat induced by acetylcholine (10 μM).

FIG. 13 shows the effect of accumulated concentrations of L294 derivatives on the contracture of the tracheal rings of a rat. (* indicates p<0.05 when compared to the control). The numbers in brackets correspond to the number of experiments carried out.

FIG. 14 shows the records of EDL and SOL muscle twitches in mice in the absence (Control) and presence of L786 (50 μM) and thirty minutes after bathing the preparations with Krebs solution. Note the alteration in the speed of the record.

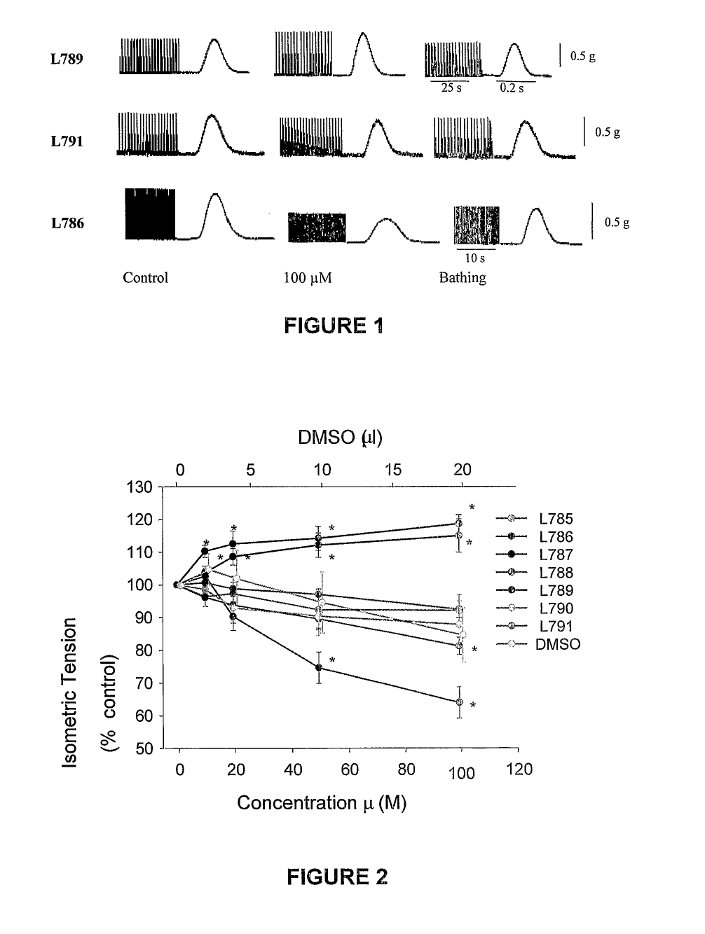

### SUMMARY OF THE INVENTION

One of the purposes of this invention is the presentation of thienyl-acylhydrazone derivatives that can cause selective muscle relaxation. More specifically, these derivatives are selected for the cardiac and/or smooth and/or skeletal muscles.

An additional purpose of this invention is the thienyl-acylhydrazone derivatives directly related to the general formula (I) below:

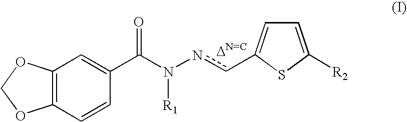

where:

R1 may be H, C1-C6 alkyl, C1-C6 alkenyl or arylalkyl, with these radicals being optionally substituted, unsaturated and/or ramified;

Δ^{N═C} refers to the bond between the nitrogen and the carbon, which bond may be single or double, provided that when the bond is single, the nitrogen forms another single bond with a hydrogen;

R2 may be H, C1-C6 alkyl, halogen, NO₂, with the radicals being optionally substituted, unsaturated and/or ramified; and their pharmaceutically acceptable salts, derivatives and/or solvates.

An additional purpose of this invention consists of pharmaceutical compositions containing such derivatives and their use in animals, preferably humans. More specifically, these compositions are intended for the relaxation of the cardiac and/or smooth and/or skeletal muscles.

### DETAILED DESCRIPTION OF THE INVENTION

As can be clearly seen, the compounds in this invention are directly related to the LASSBio-294 compound (L294) presented in Document WO 00/78754 and derivatives. According to the descriptive report for this reference, the purpose of this invention consists only of compounds with positive inotropic activity, meaning those able to increase muscle cell contractility. The derivatives described here differ from these compounds precisely because they present negative inotropic activities, in addition to tissue selectivity.

The examples described here are not intended to limit the invention, and are merely illustrative.

For the purposes of this invention, muscle tissue is taken as being the set of tissues formed by the cardiac, smooth and skeletal muscles. Still for the purposes of this invention, the phrase “L294 analogs” encompasses the L785, L786, L787, L788, L790 and L791 compounds.

The thienyl-acylhydrazone derivatives encompassed by this invention are compounds directly related to the general formula (I) below:

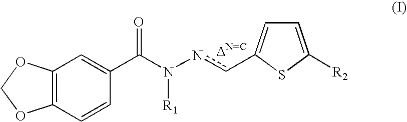

where:

R1 may be H, C1-C6 alkyl, C1-C6 alkenyl or arylalkyl, with these radicals being optionally substituted, unsaturated and/or ramified;

Δ^{N═C} refers to the bond between the nitrogen and the carbon, which bond may be single or double, provided that when the bond is single, the nitrogen forms another single bond with a hydrogen;

R2 may be H, C1-C6 alkyl, halogen, NO₂, with the radicals being optionally substituted, unsaturated and/or ramified; and the pharmaceutically acceptable salts, derivatives and/or solvates thereof.

The derivatives encompassed by this invention are endowed with tissue selectivity, acting preferentially on the muscle tissue. Moreover, the compounds encompassed by this invention present selectivity among the muscle tissues, acting on at least one of the tissues in the group selected for assessment, consisting of cardiac muscle, vascular smooth muscle and skeletal muscle.

The derivatives encompassed by this invention may be prepared in accordance with state-of-the-art chemical procedures that are known and described.

The preferential compounds of this invention, the L294 analogs follow the general formula (I) and its substitute elements, corresponding to Table 1 below.

TABLE 1
Preferential Compounds of the Invention

| Derivative | R1 | Δ^{N═C} | R2 |
|---|---|---|---|
| LASSBio 785 (L785) | CH₃— | Yes | H |
| LASSBio 786 (L786) | C₆H₅CH₂— | Yes | H |
| LASSBio 787 (L787) | H | Yes | CH₃ |
| LASSBio 788 (L788) | CH2═CHCH₂— | Yes | H |
| LASSBio 790 (L790) | H | Yes | NO₂ |
| LASSBio 791 (L791) | H | No | H |

The preferential compounds of the invention are selected from the group that consists of:
1,3-Benzodioxol-5-carboxylic acid, N-methyl-N′-[1-thiophene-2-yl-meth-(E)-ylidene]-hydrazide (L785)
1,3-Benzodioxol-5-carboxylic acid, N-benzyl-N′-[1-thiophene-2-yl-meth-(E)-ylidene]-hydrazide (L786)
1,3-Benzodioxol-5-carboxylic acid, [1-(5-methyl-thiophene-2-yl)-meth-(E)-ylidene]-hydrazide (L787)
1,3-Benzodioxol-5-carboxylic acid, N′-[1-thiophene-2-yl-meth-(E)-ylidene]-N-allyl-hydrazide (L788)
1,3-Benzodioxol-5-carboxylic acid, [1-(5-nitro-thiophene-2-yl)-meth-(E)-ylidene]-hydrazide (L790)
1,3-Benzodioxol-5-carboxylic acid, N′-thiophene-2-ylmethyl-hydrazide (L791)

In order to assess the biological activity of the compounds of this invention, several experiments were carried out as shown in the following scheme:

1) Cardiac muscle
a. Isometric tension in papillary muscle
b. Isometric tension in ventricular bundles

2) Vascular smooth muscle
a. Vascular
   i. Isometric tension in aorta artery—induction by phenylephrine (with endothelium)
   ii. Isometric tension in aorta artery—induction by KCl (with endothelium)
   iii. Isometric tension in aorta artery—induction by phenylephrine (without endothelium)
   iv. Isometric tension in aorta artery—induction by KCl (without endothelium)
b. Corpus Cavenosa
   i. Contracture induced by phenylephrine
c. Tracheal
   i. Contracture induced by acetylcholine

3) Skeletal muscle
a. Amplitude of twitches induced electrically in rapid fibers
b. Amplitude of twitches induced electrically in slow fibers

Once the experiments have been carried out, the findings were subject to statistical analyses being expressed in average ±SEM as a percentage of the control value, and graphs were drawn up using the Sigma Plot 5.0 program to represent the data obtained. For the statistical analysis of the effects on the different muscle tissues, the difference was assessed among the concentrations of the same derivative, using the “t” Student test. In order to ascertain the difference among the derivatives, the one-way ANOVA test was used. The differences were considered as significant for p<0.05. The Cl₅₀ was expressed as an average ±SEM of the values obtained for each experiment through non-linear regression based on the y=yₒ+ae^{-bx} equation.

Laboratory Trials

Cardiac Muscle

Experiment Protocol

After anesthesia with ethyl ether, male Wistar rats weighing between 240-280 grams were sacrificed through cervical dislocation. The heart was removed rapidly and the papillary muscle and the ventricular bundles were dissected in Tyrode's solution, whose composition is given in Table 2 below, at room temperature. The muscles were placed in vertical chambers with a capacity of 10 mL and fixed at their extremities: the lower portion to a hook located in the stimulation electrode fixation rod and the upper portion connected to a power transducer (Grass FT03) for recording the muscle twitches.

TABLE 2
Tyrode's solution composition in
mM. pH adjusted to 7.4 with KOH 5M

| Compounds | Concentration (mM) |
|---|---|
| NaCl | 130.0 |
| KCl | 5.0 |
| MgCl₂ | 1.0 |
| CaCl₂ | 2.5 |
| NaHCO₃ | 24.0 |
| Na₂HPO₄ | 0.9 |
| Dextrose | 5.6 |

The muscles were stimulated electrically with supra-maximum voltage (50-60 V) through a stimulator (Grass S88) at a frequency of 1.0 Hz. The signals captured by the transducer were amplified (Cyberamp 380, Axon Instruments) and the isometric tension was digitized (Digidata 1322A, Axon Instruments) and stored on a computer. The screening and analysis of the experiment was handled through the Axoscope 8.0 program (Axon Instruments) (Sudo & cols., 2001). The chamber solution was bubbled continuously with a carbogenic mixture (95% O₂/5% CO₂) and kept at a temperature of 37° C.

After the stabilization of the muscle twitches (approximately 30 min) the records were carried out in the absence (control) and presence of rising concentrations (10 to 100 μM) of the different L294 analogs, diluted in DMSO. The records were carried out in pairs (two papillary and two ventricle) thus allowing the control experiments to be carried out in one of the pairs, meaning with rising quantities of DMSO equivalent to those used to dilute the substances. The reversibility of the effect of the substances was investigated at the end of each experiment, noting the recovery of the muscle twitches after bathing the preparation with Tyrode's solution.

Findings

FIG. 21 shows the typical records of papillary muscle twitches in the absence and presence of 100 μM of L789, L791 and L786.

This effect was fully reverted after bathing the preparation with Tyrode's solution. The response diversity of the L294 analogs in the heart can be seen in the two following records. Thus, the same concentration of (100 μM) of L791 did not significantly alter the contractile response of the papillary muscle. On the other hand, the L786 sharply reduced the amplitude of the muscle twitches, with this effect being partially reverted through bathing the preparation.

The dose-response curve relating the isometric tension of the papillary muscle to the rising concentration of the L294 analogs is shown in FIG. 2. The L789 significantly increased the amplitude of the muscle twitches in relation to the control. The maximum effect with this analog was achieved at a concentration of 100 μM (120.5±3.7% of the control, n=6, p<0.05). In contrast, L786 and L788 reduced the amplitude of the papillary muscle twitches in a concentration-dependent manner, dropping from 64.1±5.9% and 81.2±2.6% of the control respectively in the 100 μM concentration. Still in the papillary muscle, the maximum alterations to the twitches caused by L785 (86.5±4.0% of the control, n=6), the L787 (115.0±5.0% of the control, n=6), L790 (87.8±5.6% of the control, n=6) and L791 (92.4±2.5% of the control, n=6) were not significant. The DMSO added to the solution in the same volume as the substances were diluted did not significantly modify the muscle twitch amplitude.

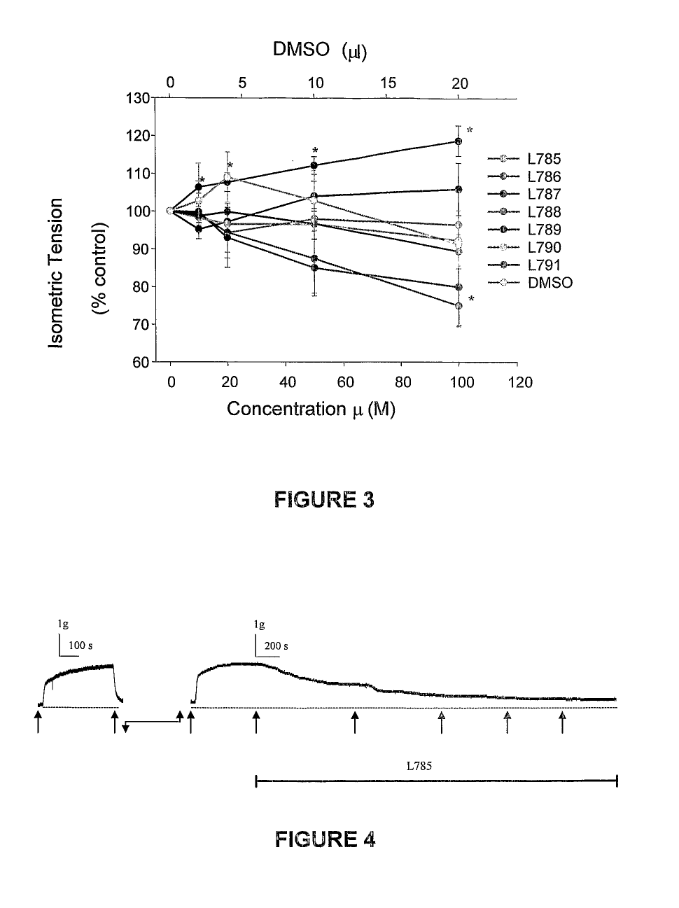

As shown in FIG. 3, similar findings were obtained for ventricular bundles where the L789 (100 μM) also significantly increased the muscle twitches to 118.8±4.0% of the control (p<0.05). The L786 and L788 (100 μM) reduced muscle contractility to 79.9±10.6% (p<0.01) and 74.9±5.1% (p<0.05) of the control respectively. The other derivatives tested in this preparation did not cause alterations greater than 10%, namely L785, L787, L790 and L791 modified the muscular response at 96.5±3.8%, 105±6.9%, 92.3±1.6% and 89.4±4.5% of the control respectively.

The reversal of the decrease or increase in the contractility of the cardiac musculature was assessed after the perfusion of the nutrient solution without the derivative being tested, for thirty minutes. The recovery of the muscle twitches was complete for most of the derivatives, except for the L786, which still remained at 79.7±4.6% of the control (p<0.05) after bathing.

Vascular Smooth Muscle—Aorta Artery

Experiment Protocol

Male Wistar rats weighing between 240-280 grams were sacrificed through dislocation of the cervical column under anesthesia using ethyl ether. The aorta artery was removed and transferred to a Petri dish containing modified Tyrode's solution whose composition is described in Table 3 below, where the vessel was cleaned and the connective tissue was removed. According to the scheduled Experiment Protocol, the vascular endothelium was removed mechanically with the assistance of a polyethylene rod. The artery was divided into rings with a width of 2-3 mm. Two hooks were placed in the wall of the vessel in order to record the tension generated by the muscle in a transversal direction. To do so, one of the hooks was tied to a fixed point in the chamber and the other to a power transducer (FT 03) for recording the isometric tension. The tension generated by the muscle was transformed into a digitized electric signal (Digidata 1322A) and stored in a computer for subsequent analysis using the Axoscope 8.0 program (Axon Instruments, Inc). The chamber was filled with Krebs solution, oxygenated and kept at a temperature of 37° C.

TABLE 3
Tyrode's solution composition in mM. The solution
was bubbled continuously in a 95% O₂/5% CO₂ mixture
with the pH adjusted to 7.4. The temperature of the
experimental medium was kept at 37° C.

| Compounds | Concentration (mM) |
|---|---|
| NaCl | 120.0 |
| KCl | 5.9 |
| MgCl₂ | 1.2 |
| CaCl₂ | 2.5 |
| NaHCO₃ | 24.0 |
| Na₂HPO₄ | 0.9 |
| Dextrose | 5.6 |

After stabilizing the preparation, approximately 120 minutes, the Experiment Protocol began. Initially, in order to identify the presence or absence of the vascular endothelium, the preparation was exposed to phenylephrine (10 μM) and when the contracture in response to this substance was stabilized, acetylcholine (10 μM) was added to the nutrient solution. Relaxation of over 80% in response to the acetylcholine indicated the full integrity of the endothelium and relaxation of less than 10% indicated the absence of endothelium. The tissues in which the relaxation intensity fell between >10% and <80% were discarded. In order to investigate the vasodilator effect of the different L294 analogs, they were added to the experiment chambers in rising concentration after the contracture stabilization of the vessel induced by phenylephrine. The tension registered after treatment with each of the L294 analog concentrations was normalized as a function of the amplitude of the contracture triggered by phenylephrine. For the purpose of comparing the potency of some of the L294 analogs, the Cl₅₀ was determined for each experiment using the y=yₒ+ae^{-bx} equation and their mean was used for the statistical analysis.

Findings

Effects on the Contracture Induced by Phenylephrine in Preparation with Endothelium

FIG. 4 shows a typical record of an experiment carried out with rat aorta rings with a whole endothelium. After the stabilization of the contracture in response to phenylephrine (10 μM), 10 μM of acetylcholine (Ach) were added to the experiment chamber. The relaxation caused by the Ach was superior to 80% of the control, thus demonstrating the preservation of the vascular endothelium. The Ach effect was fully reversible as the phenylephrine caused a contracture whose intensity was equal to that at the start of the experiment, thirty minutes after bathing the preparation. The L785 analog reduced the phenylephrine contracture in a dose-dependent manner, reaching its maximum effect at a concentration of 100 μM.

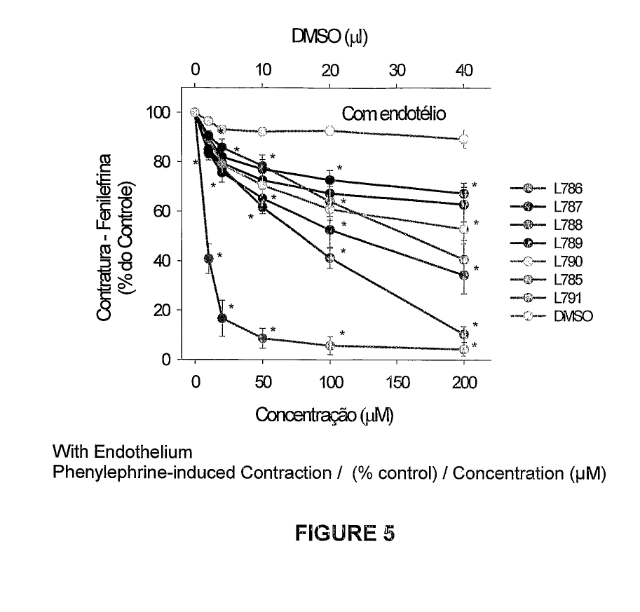

A protocol similar to that used in FIG. 4 was repeated for the other substances studied. As shown in FIG. 5, it is clear that all the L294 analogs have effects similar to that of the L785, although with significant differences in potency and efficacy among them. The L785 and L788 were the most efficacious derivatives, reducing the amplitude of the contracture in rings with whole endothelium to 4.4±2.8% (n=6) and 10.4±3.2% (n=6) of the control respectively.

However, the maximum inhibitory effect induced by the L785 was achieved with 50 μM, a concentration four times less than that of the L788 for causing inhibition of equivalent intensity. The maximum inhibition levels observed with the other derivatives were 34.5±7.6% (n=6), 37.1±7.0% (n=6), 32.7±4.2% (n=6), 47.0±5.9% (n=6) and 59.2±7.8% for L786, L787, L789, L790 and L791 respectively.

The comparative potency among the L294 analogs in this preparation is shown in Table 5 below. Note that the Cl₅₀ of the L785 is almost seven times less than that of the L788 and almost thirty times less than that of the L787, showing the marked potency of this substance. The analog L789 is low potency, not causing significant vascular relaxation, and consequently did not allow this determination of the Cl₅₀.

TABLE 5
CI₅₀ of the L294 analogs in phenylephrine-induced contracture (10
μM) in aorta rings with preserved endothelium. The CI₅₀ was
determined for each experiment and the data in the Table represent
the average ± SEM for six experiments. ND shows the situation in
which the CI₅₀ could not be determined because of the low potency
of the analog for inhibiting the phenylephrine-induced contracture.
Inhibition of the Phenylephrine-Induced Contracture - with Endothelium

| L294 Analogs | CI50 (μM) Average ± SEM |
|---|---|
| L785 | 10.2 ± 0.5 |
| L788 | 67.9 ± 6.5 |
| L786 | 134.1 ± 31.0 |
| L791 | 172.8 ± 26.7 |
| L790 | 216.0 ± 39.3 |
| L787 | 293.0 ± 76.0 |
| L789 | NG |

Effects on KCl-Induced Contracture in Preparation with Endothelium

The effects of the L294 analogs were investigated in rat aorta rings with preserved endothelium whose contracture was induced by increasing the extra-cellular concentration of KCl to 40 mM. Of the seven L294 analogs, this test was carried out only with the L785, L786, L789 and L790, in this group of experiments.

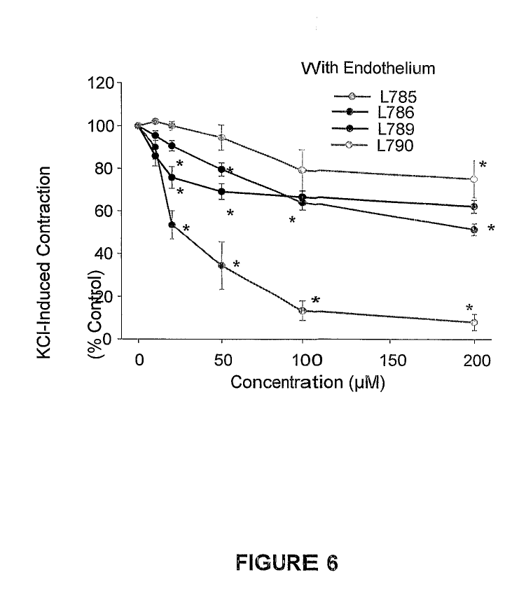

The selection criteria for the L294 analogs to be tested in the KCl-induced contracture were based on the findings obtained for the cardiac muscles and the relaxation caused in vessels of the phenylephrine induced contracture. Thus, the L785 and L786 were the most powerful for causing vascular relaxation, the L789 for the positive inotropic cardiac effect and the L790 for the fact that it is an inert analog for both the heart and the vessel. The inhibition of the contracture caused by L790 was insignificant up to 100 μM, and this was thus reduced to 74.9±8.8% (p<0.05 in relation to the control) at a concentration of 200 μM. The L789 and L786 analogs reduced the KCL contracture to 61.9±3.0% (p<0.05 in relation to the control) and 51.1±2.8% (p<0.05 in relation to the control) of the control respectively. Once again, L785 was the most effective analog, reducing the amplitude of the contracture to 8.1±3.8% of the control (p<0.05), as shown in FIG. 6.

As shown in Table 6 the Cl₅₀ with L785 reached 34.1±6.3 μM while with the L786 this reached 127.1±13.7 μM. Due to low potency, the Cl₅₀ of the L789 and L790 cannot be determined.

TABLE 6
CI₅₀ of the L294 analogs in the KCl-induced contracture
(40 mM) in aorta rings with preserved endothelium. The CI₅₀ was
determined for each experiment and the data in the Table represent
the average ± SEM for these six experiments. ND indicates situations
in which the CI₅₀ cannot be determined as a result of
the reduced potency of the analog for inhibiting the contracture
induced by increasing the extra-cellular concentration of K⁺.
Inhibition of the KCl-Induced Contracture - with Endothelium

| L294 analogs | CI₅₀ (μM) Average ± SEM |
|---|---|
| L785 | 34.1 ± 6.3 |
| L786 | 127.1 ± 13.7 |
| L789 | ND |
| L790 | ND |

Effects on the Phenylephrine-Induced Contracture in a Preparation without Endothelium

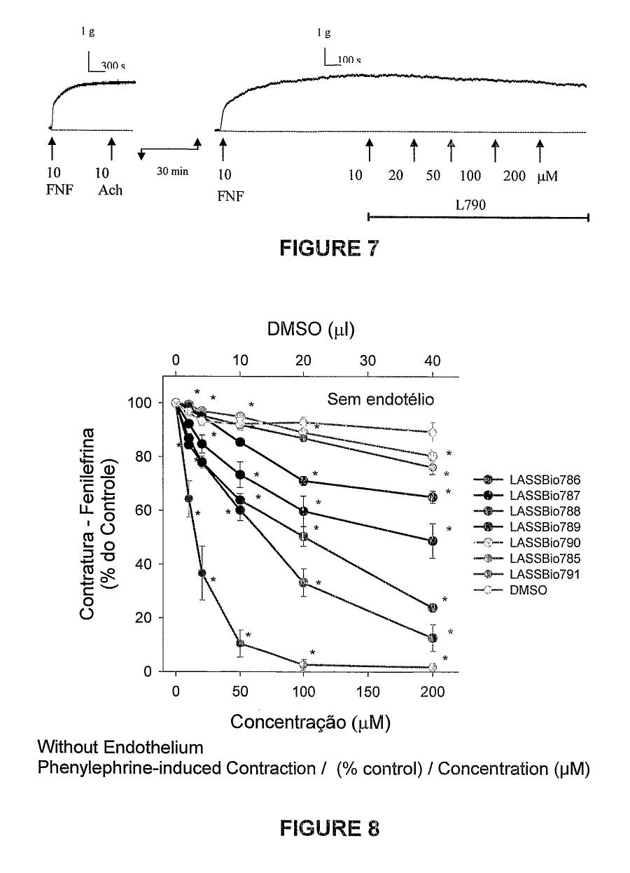

The importance of the presence of vascular endothelium in the vasodilatory action of the L294 analogs was assessed in experiments in which these cells were removed mechanically. The records in FIG. 7 demonstrated the absence of the vascular endothelium through the non-relaxation of the vessel in the presence of Ach. Additionally, the replicability of the phenylephrine-induced contractures was demonstrated, even after bathing the preparation and in the absence of the relaxing effect of the L790. When the findings of the experiments without the endothelium were analyzed as a whole, the absence of the L790 effect was confirmed. Similarly, structural modifications to L791 did not result in any vasodilatory activity in this preparation (FIG. 8). The maximum relaxation caused by the L787 and L789 analogs was less than 50% of the control, and the other analogs caused relaxation in the following decreasing order of efficacy: L785>L788>L786. Once again, attention is drawn to the marked efficiency of the L785 and L788 analogs. With L785, relaxation was almost total with 100 μM of the substance.

Potency among the L294 analogs followed the same order of efficacy. As shown in Table 7 below, the Cl₅₀ of the L785 is notably well below that of the other substances. The intermediate group includes L788 and L786, and among those causing effects, the least powerful were L789 and L787. The L791 and L790 analogs lacked vasodilatory action (Table 7).

TABLE 7
CI₅₀ of the L294 analogs in phenylephrine-induced contracture (10
μM) in aorta rings with no preserved endothelium. The CI₅₀ was
determined for each experiment and the data in the Table represent
the average ± SEM for six experiments. ND indicates situations
in which the CI₅₀ cannot be determined as a function of the reduced
potency of the analog in inhibiting the phenylephrine-induced
contracture. Inhibition of the Phenylephrine-Induced Contracture -
Without Endothelium

| L294 analogs | CI₅₀ (MM) Average ± SEM |
|---|---|
| L785 | 18.5 ± 3.6 |
| L788 | 65.7 ± 8.0 |
| L786 | 86.2 ± 7.1 |
| L789 | 196.1 ± 39.7 |
| L787 | 273.4 ± 22.0 |
| L791 | NG |
| L790 | NG |

Effects on Contracture Induced by KCl in Preparation without Endothelium

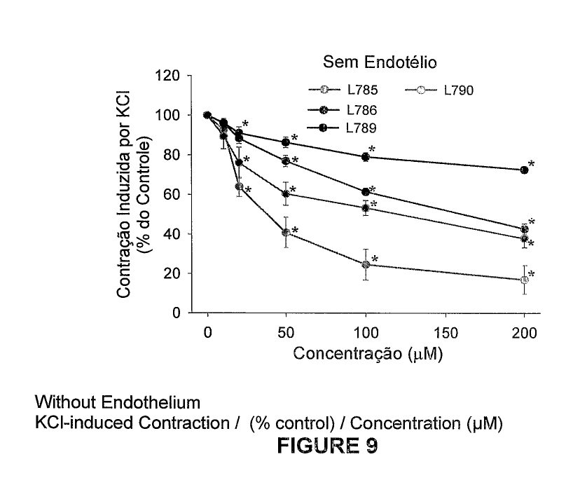

The L294 analogs also reversed the contracture caused by K⁺ in a preparation without endothelium. There was no difference in efficacy between the L786 and L790 that constitute an intermediate group (FIG. 9). The analog with the lowest efficiency was L789 with L785 having the highest efficacy. In contrast to contracture force by phenylephrine, L785 was unable to completely reverse the contracture caused by K⁺, even at the highest concentration (200 μM), of this preparation.

Table 8 presents the comparative Cl₅₀ among the L294 analogs in the contracture induced by K⁺ in the preparation of aorta with no endothelium. It is noted that the L785 was the most powerful analog (Cl₅₀=40.5±9.5 μM) followed by L790 (123.0±17.3 μM) and L786 (151.4±7.6 μM). The L789 was the least powerful (Cl₅₀=394.0±25.4 μM).

TABLE 8
CI₅₀ of the L294 analogs in the contracture induced by KCl (40 mM) in
aorta rings without preserving the endothelium. The CI₅₀ was determined
for each experiment and the data in the Table represent the average ± SEM
for six experiments. ND indicates the situation in which CI₅₀ could
not be determined due to the limited power of the analog to inhibit
the contracture induced by the increase in the extracellular KCl.
Inhibition of Contracture Induced by KCl - Without Endothelium

| L294 analogs | CI50 (μM) Average ± SEM |
|---|---|
| L785 | 40.5 ± 9.5 |
| L786 | 151.4 ± 7.6 |
| L789 | 394.0 ± 25.4 |
| L790 | NG |

Corpus Cavernosa

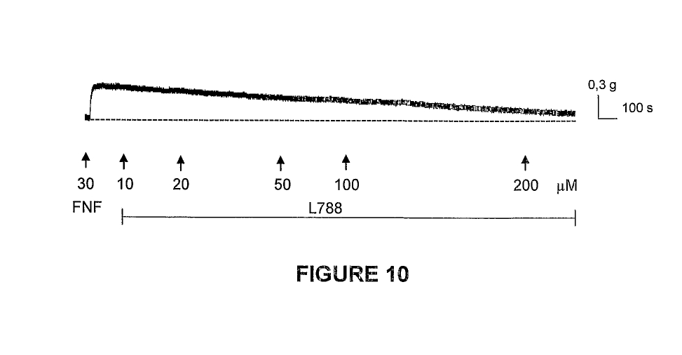

The L294 derivatives that proved more powerful for causing relaxation of the vascular smooth musculature such as L785, L786 and L788, and another derivative unable to relax the vascular smooth musculature, L790 were initially assessed in the contractile response of the smooth musculature of a corpus cavernosa of a male guinea pig. FIG. 10 shows a typical record of the phenylephrine-induced contracture (30 μM) in the corpus cavernosa of a male guinea pig, followed by exposure to rising cumulative concentrations of L788 up to 200 μM. It is noted that L788 concentration-dependent inhibition occurred for the contractile response of the corpus cavernosa.

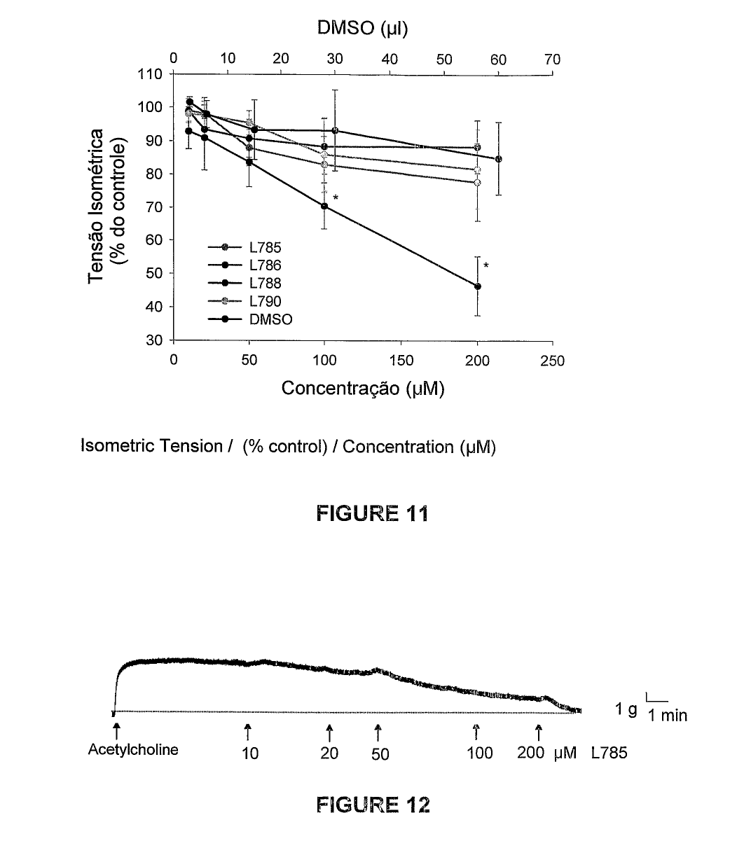

This same Protocol Experiment was repeated for the other derivatives being tested and for the solvent separately (FIG. 11). It was noted that only L788 caused significant reaction, where the amplitude of the contracture at a concentration of 200 μM was 46.5±8.8% of the control. Meanwhile, the other derivatives did not cause significant effects on the amplitude of the contractile response of the corpus cavernosa. The effect of the solvent (DMSO) was tested at volumes corresponding to those used for the derivatives, and was unable to cause any significant alterations to the contractility of this tissue.

Trachea

A preparation of trachea rings is used for observing the probable action of these powerful vasodilators in other smooth musculature tissues. FIG. 12 presents the typical record of the acetylcholine-induced trachea rings contracture (Ach, 10 μM) followed by exposure to rising cumulative concentrations of the L785 derivative up to 200 μM. A dose-dependent reduction may be noted in the amplitude of the acetylcholine-induced contracture.

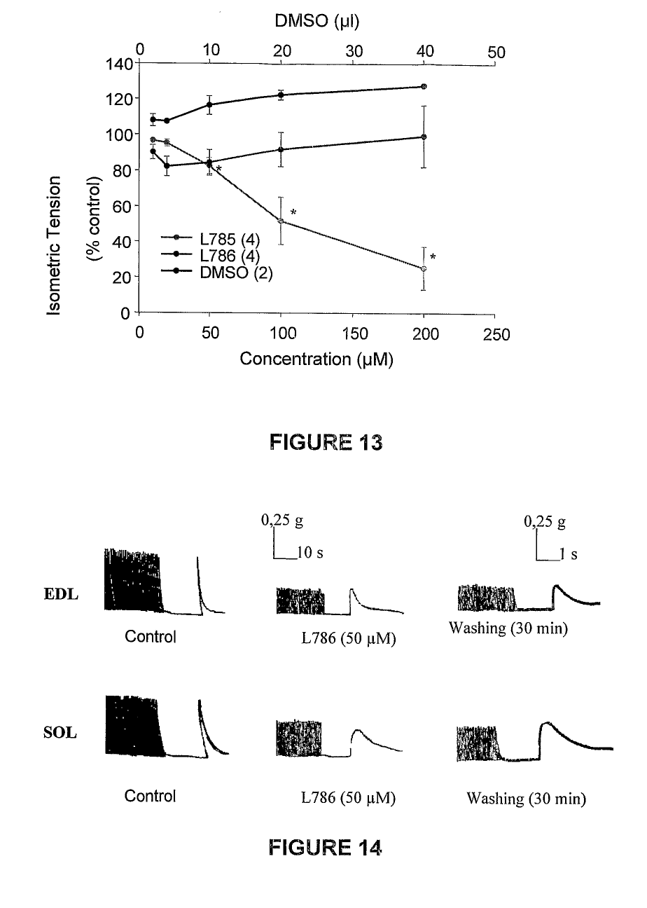

The same protocol was used for the L786 derivative and the solvent (FIG. 13). It is noted that the L785 caused a significant dose-dependent muscle relaxation effect. At a concentration of 200 μM, L786 reduced the amplitude of the contracture to 25.3±12.1% of the control. The same effect was not observed with L786. It should be stressed that the solvent used (DMSO) did not present a relaxation effect, but to the contrary caused a slight increase in the contracture amplitude (127.5±0.9% of the control) at the volume corresponding to the 200 μM concentration of the derivatives.

Skeletal Muscle

Experiment Protocol

Male mice weighing 22-28 g were sacrificed through cervical dislocation and the extensor digitorium longus (EDL) and soleus (SOL) muscles were dissected in a Ringer-Krebs solution, whose composition is given in Table 9, at room temperature. The muscles were placed in vertical chambers with a capacity of 10 ml, and were fixed by their ends to the stimulation electrode fixation rod and a power transducer (Grass FT03) for registering muscle twitches. The chamber solution was bubbled continuously with a carbogenic mixture (95% O₂/5% CO₂) and kept at a temperature of 37° C. The muscles were stimulated electrically through a stimulator (Grass S88) at a frequency of 0.2 Hz, at 37° C. After the stabilization of the muscle twitches (±30 minutes) a control record was taken in the absence of the drug, and subsequently rising concentrations (from 10 μM to 200 μM) of the L294 derivatives were added every ten minutes, diluted in DMSO. The reversibility of the preparation was noted at the end of the experiment through bathing with nutrient solution. The signals recorded by the transducer were amplified (Cyberamp) and the isometric tension was digitized (Digidata 1322A) and stored on a computer. The screening and analysis of the experiment was handled through the use of the Axoscope 8.0 program. (Zapata-Sudo, 2003).

TABLE 9
Composition of the Ringer-Krebs solution
in mM. pH adjusted to 7.4 with KOH 5M.

| Compounds | Concentration (mM) |
|---|---|
| NaCl | 135.0 |
| KCl | 5.0 |
| MgCl₂ | 1.0 |
| CaCl₂ | 2.0 |
| NaHCO₃ | 15.0 |
| Na₂HPO₄ | 1.0 |
| Dextrose | 11.0 |

Findings

A measurement of the electrically-induced twitch amplitude was selected to assist the effects of the L294 analogs on the skeletal muscle inotropism. One muscle consisting of fast fibers (extensor digitorium longus-EDL) and the other of slow fibers (soleus-SOL) taken from mice were selected for this purpose. These typical experiments at two recording speeds were carried out on the SOL and EDL muscles, presented in FIG. 14. Differences in the timing of the twitches between the fast and slow muscles are shown at the faster recording speed. The administration of 50 μM of L786 clearly caused a reduction in the amplitude and an extension in the duration of the muscle twitches in both the EDL and the SOL muscles. Washing the preparation with Krebs solution caused a partial reversion of the effects induced by the L786.

The same protocol was repeated for the other analogs and the findings are presented in FIGS. 19 and 20. A difference was noted in the effect of the derivatives between the muscles. The L786 proved more effective in reducing the amplitude of the EDL muscle twitch (28.7±9.9% of the control) than the SOL muscle (61.9±5.5% of the control). The L785 presented equivalent efficacy, reducing the EDL and SOL muscle contraction to 31.6±4.7% and 28.4±6.6% of the control respectively, stressing that this was the derivative that was most efficacious for the SOL muscle. In the latter, the L787 and L788 derivatives were noteworthy for reducing the amplitude to 47.6±4.2% and 45.1±5.1% of the control respectively, while the other derivatives, L789, L790 and L791, caused reductions of under 50%, namely 59.4±3.4%, 71.3±5.2% and 69.3±4.8% of the control respectively.

In the EDL muscle, these three latter derivatives caused larger reductions at 38.9±4.8%, 52.2±3.4% and 45.8±6.9% respectively, and the L787 and L788 derivatives reduced the amplitude to 37.8±3.5% and 47.6±5.4% of the control respectively. However, this effect on the EDL may be attributed in part to the solvent, with this fact not being observed in the SOL.

The inhibition of the skeletal muscle twitches caused by the L294 analogs, could not be totally reversed through bathing. As presented in Table 10, most of the analogs caused irreversible effects thirty minutes after bathing. The effects of some analogs—such as L791 in the EDL and L787 in the SOL—could be partially reversed.

TABLE 10
Reversal of the effects of the L294 analogs on
the amplitude of the skeletal muscle twitches.

| | EDL | | SOL | |
|---|---|---|---|---|
| | 200 μM | 30 min-Washing | 200 μM | 30 min-Washing |
| Deriv- | (% of the | (% of the | (% of the | (% of the |
| atives | control) | control) | control) | control) |
| L785 | 31.6 ± 4.7 | 36.4 ± 4.5 | 28.4 ± 6.6 | 37.9 ± 9.6 |
| L786 | 28.7 ± 9.9 | NG | 61.9 ± 5.5 | 77.0 ± 10.3 |
| L787 | 37.8 ± 3.5 | 38.6 ± 2.5 | 47.6 ± 4.2 | 76.0 ± 4.6 |
| L788 | 47.6 ± 5.4 | 26.8 ± 2.3 | 45.1 ± 5.1 | 37.8 ± 4.7 |
| L789 | 38.9 ± 4.8 | 42.2 ± 6.4 | 59.4 ± 3.4 | 60.6 ± 5.5 |
| L790 | 52.2 ± 3.8 | 49.2 ± 5.1 | 71.3 ± 5.2 | 70.8 ± 7.7 |
| L791 | 45.9 ± 6.9 | 71.5 ± 19.0 | 69.3 ± 4.8 | 74.4 ± 13.8 |

The compounds of the invention may be administered in a variety of dosage forms, such as orally as tablets, capsules, sugar or tablets covered with film, liquid solutions or suspensions; rectally as suppositories; parenterally, through intramuscular injection or by intravenous and/or intrathecal and/or intraspinal injection or infusion.

This invention also includes pharmaceutical compositions that encompass the formula (I) compound or the pharmaceutically acceptable salts thereof, in association with pharmaceutically acceptable excipient.

The pharmaceutical compositions containing the compounds of the invention are normally prepared according to conventional methods and are administered in appropriate pharmaceutical manners.

1. Substance characterized by having a structure in compliance with the general formula (I): 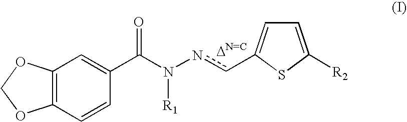
wherein:
R1 is H, C1-C6 alkyl, C1-C6 alkenyl or arylalkyl, with these radicals being optionally substituted, unsaturated and/or ramified;
Δ^{N═C} refers to the bond between the nitrogen and the carbon, which is a single or a double bond, provided that when the bond is single, the nitrogen forms another single bond with hydrogen;
R2 is H, C1-C6 alkyl, halogen, or NO₂, with the radicals being optionally substituted, unsaturated and/or ramified; and their pharmaceutically acceptable salts, derivatives and/or solvates,
with the proviso that when R2 is H and Δ^{N═C} is a double bond, R1 is arylalkyl or C1-C6 alkenyl, with these radicals being optionally substituted and/or ramified,
wherein the substance is useful for selective muscle relaxation.

2. Substance, according to claim 1, characterized by being selected from the group consisting of:
1,3-Benzodioxol-5-carboxylic acid, N-benzyl-N′-[1-thiophene-2-yl-meth-(E)-ylidene]-hydrazide;
1,3-Benzodioxol-5-carboxylic acid, [1-(5-methyl-thiophene-2-yl)-meth-(E)-ylidene]-hydrazide;
1,3-Benzodioxol-5-carboxylic acid, N′-[1-thiophene-2-yl-meth-(E)-ylidene]-N-allyl-hydrazide
1,3-Benzodioxole-5-carboxylic acid, [1-(5-nitro-thiophene-2-yl)-meth-(E)-ylidene]-hydrazide; and
1,3-Benzodioxole-5-carboxylic acid, N′-thiophene-2-ylmethyl-hydrazide,
wherein the substance is useful for selective muscle relaxation.

3. (canceled)

4. Substance according to claim 1, wherein the muscle is selected from the group consisting of smooth muscle, cardiac muscle, skeletal muscle, and combinations thereof.

5. Pharmaceutical composition characterized by comprising as an active ingredient at least one substance of the formula (I): 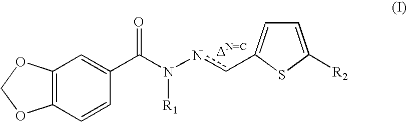
wherein:
R1 is H, C1-C6 alkyl, C1-C6 alkenyl or arylalkyl, with these radicals being optionally substituted, unsaturated and/or ramified;
Δ^{N═C} refers to the bond between the nitrogen and the carbon, which is a single or a double, provided that when the bond is single, the nitrogen forms another single bond with hydrogen; and
R2 is H, C1-C6 alkyl, halogen, or NO₂, with the radicals being optionally substituted, unsaturated and/or ramified; and their pharmaceutically acceptable salts, derivatives and/or solvates,
with the proviso that when R2 is H and Δ^{N═C} is a double bond, R1 is arylalkyl or C1-C6 alkenyl, with these radicals being optionally substituted and/or ramified, and a pharmaceutically acceptable vehicle,
wherein the pharmaceutical composition is useful for selective muscle relaxation.

6. Pharmaceutical composition according to claim 5, characterized by wherein the active ingredient is selected from the group consisting of:
1,3-Benzodioxole-5-carboxylic acid, N-benzyl-N′-[1-thiophene-2-yl-meth-(E)-ylidene]-hydrazide;
1,3-Benzodioxol-5-carboxylic acid, [1-(5-methyl-thiophene-2-yl)-meth-(E)-ylidene]-hydrazide;
1,3-Benzodioxol-5-carboxylic acid, N′-[1-thiophene-2-yl-meth-(E)-ylidene]-N-allyl-hydrazide;
1,3-Benzodioxol-5-carboxylic acid, [1-(5-nitro-thiophene-2-yl)-meth-(E)-ylidene]-hydrazide;
1,3-Benzodioxol-5-carboxylic acid, N′-thiophene-2-ylmethyl-hydrazide; and combinations thereof,
wherein the pharmaceutical composition is useful for selective muscle relaxation.

7. (canceled)

8. Pharmaceutical composition according to claim 5, wherein the muscle is selected from the group consisting of smooth muscle, cardiac muscle, skeletal muscle, and combinations thereof.

9. Use of an active ingredient having the formula (I): 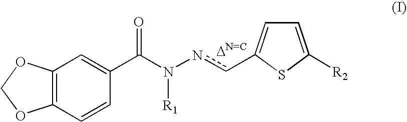
wherein:
R1 is H, C1-C6 alkyl, C1-C6 alkenyl or arylalkyl, with these radicals being optionally substituted, unsaturated and/or ramified;
Δ^{N═C} refers to the bond between the nitrogen and the carbon, which is a single bond or a double bond, provided that when the bond is a single bond, the nitrogen forms another single bond with hydrogen;
R2 is H, C1-C6 alkyl, halogen, or NO₂, with the radicals being optionally substituted, unsaturated and/or ramified; and their pharmaceutically acceptable salts, derivatives and/or solvates,
with the proviso that when R2 is H and Δ^{N═C} is a double bond, R1 is arylalkyl or C1-C6 alkenyl, with these radicals being optionally substituted and/or ramified,
wherein the active ingredient is employed in the preparation of a medicament useful in the treatment of diseases associated to muscular tissue.

10. (canceled)

11. (canceled)

12. Substance according to claim 2, wherein the muscle is selected from the group consisting of smooth muscle, cardiac muscle, skeletal muscle, and combinations thereof.

13. Pharmaceutical composition according to claim 6, wherein the muscle is selected from the group consisting of smooth muscle, cardiac muscle, skeletal muscle, and combinations thereof.

14. Substance characterized by having a structure in compliance with the general formula (I): 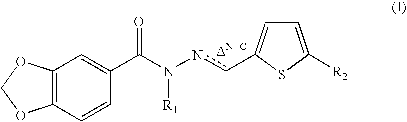
wherein:
R1 is CH3;
Δ^{N═C} refers to the bond between the nitrogen and the carbon, wherein the bond is double;
R2 is H;
and their pharmaceutically acceptable salts, derivatives and/or solvates wherein the substance is useful for selective muscle relaxation

15. Substance according to claim 13, wherein the muscle is selected from the group consisting of smooth muscle, cardiac muscle, skeletal muscle, and combinations thereof.

16. Pharmaceutical composition characterized by comprising as an active ingredient the substance of formula (I): 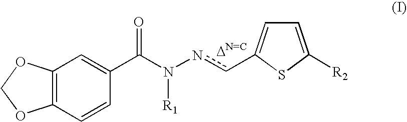
wherein:
R1 is CH3;
Δ^{N═C} refers to the bond between the nitrogen and the carbon, wherein the bond is a double bond;
R2 is H; and their pharmaceutically acceptable salts, derivatives and/or solvates, and a pharmaceutically acceptable vehicle;
wherein the pharmaceutical composition is useful for selective muscle relaxation.

17. Pharmaceutical composition according to claim 16, wherein the muscle is selected from the group consisting of smooth muscle, cardiac muscle, skeletal muscle, and combinations thereof.

18. Use of an active ingredient having formula (I): 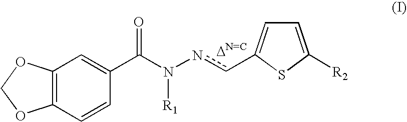
wherein:
R1 is CH3;
Δ^{N═C} refers to the bond between the nitrogen and the carbon, wherein the bond is double;
R2 is H; and their pharmaceutically acceptable salts, derivatives and/or solvates,
wherein, the active ingredient is employed in the preparation of a medicament useful in the treatment of diseases associated to muscular tissue.

19. Use of the active ingredient according to claim 9, wherein the medicament is useful for acting as a selective muscle relaxant.

20. Use of the active ingredient according to claim 9, wherein the muscular tissue is chosen from the group consisting of smooth muscle, cardiac muscle, skeletal muscle, and combinations thereof.

21. Use of the active ingredient according to claim 9, wherein the diseases associated to muscular tissue are selected from the group consisting of elevated blood pressure, angina, ischemia, erection difficulties, systemic vascular smooth muscle spasms, skeletal muscle spasms, and respiratory smooth musculature spasms.

22. Use of the active ingredient according to claim 18, wherein the medicament is useful for acting as a selective muscle relaxant.

23. Use of the active ingredient according to claim 18, wherein the muscular tissue is chosen from the group consisting of smooth muscle, cardiac muscle, skeletal muscle, and combinations thereof.

24. Use of the active ingredient according to claim 18, wherein the diseases associated to muscular tissue are selected from the group consisting of elevated blood pressure, angina, ischemia, erection difficulties, systemic vascular smooth muscle spasms, skeletal muscle spasms, and respiratory smooth musculature spasms.
